(19) United States
(12) Patent Application Publication
(10) Pub. No.: US 2021/0304897 A1
(43) Pub. Date: Sep. 30, 2021

(54) INDIVIDUAL RISK AVERSION THROUGH BIOMETRIC IDENTIFICATION

(71) Applicant: INTERNATIONAL BUSINESS MACHINES CORPORATION, Armonk, NY (US)

(72) Inventors: Kamaldeep Singh Cheema, Woodbridge (CA); Peter George Finn, Markham (CA); Moeenqaisar Mamaparo, Richmond Hill (CA); Yang Cao, Markham (CA)

(21) Appl. No.: 16/828,314

(22) Filed: Mar. 24, 2020

(51) Int. Cl. G16H 50/30; G16H 10/60; G16H 20/00

(52) U.S. Cl. CPC G16H 50/30; G16H 20/00; G16H 10/60

(57) ABSTRACT

A processor can obtain a biometric scan of a first user and a second user wherein the first user and the second user are within a predetermined geographical location. The processor can identify one or more records associated with the first user and one or more records associated with the second user based on the biometric scan. The processor can analyze the one or more records associated with the first user and the one or more records associated with the second user. The processor can determine, from the analyzing, whether a risk threshold is exceeded. The processor can display an alert to the first user and the second user.

### BACKGROUND

The present disclosure relates generally to risk aversion and more specifically, to risk aversion by biometric identification.

In today's world, people are at risk of being exposed to allergens, pathogens, or situations which may be hazardous to their health. The utilization of electronic medical records has increased the ability of healthcare providers and first responders to provide accurate and efficient treatments to individuals.

### SUMMARY

Embodiments of the present disclosure include a method, computer program product, and system for identifying health risks associated with a user. A processor can obtain a biometric scan of a first user and a second user wherein the first user and the second user are within a predetermined geographical location. The processor can identify one or more records associated with the first user and one or more records associated with the second user based on the biometric scan. The processor can analyze the one or more records associated with the first user and the one or more records associated with the second user. The processor can determine, from the analyzing, whether a risk threshold is exceeded. The processor can display an alert to the first user and the second user.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 is a functional block diagram generally depicting a biometric identification risk aversion environment, in accordance with an embodiment of the present invention.

FIG. 2 is a block diagram depicting a biometric identification risk aversion module, in accordance with an embodiment of the present invention.

FIG. 3 is a flowchart depicting operational steps of risk aversion by biometric identification, in accordance with an embodiment of the present invention.

FIG. 4 is a flowchart depicting operational steps of risk aversion by biometric identification, in accordance with an embodiment of the present invention.

FIG. 5 is a block diagram of components of a computer, suitable for executing operation of a risk aversion by biometric identification program in accordance with an embodiment of the present invention.

FIG. 6 is a block diagram depicting a cloud computing environment, in accordance with an embodiment of the present invention.

FIG. 7 is a block diagram depicting abstraction model layers, in accordance with an embodiment of the present invention.

FIG. 8 is a block diagram of an example computing environment in which illustrative embodiments of the present invention may be implemented.

FIG. 9 is a block diagram of an example natural language processing system configured to analyze user records to generate a risk aversion plan, in accordance with embodiments of the present invention.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

The embodiments depicted and described herein recognize the need to create a risk aversion plan for individuals (e.g., users) in a given location. Further, a risk aversion plan can be developed by an analysis of the health records of the individuals in a given location. The embodiments depicted and described herein recognize the benefits of creating a risk aversion plan by identifying the individuals in a given location through biometrics. Further, the risk aversion plan can allow for an individual to avoid situations that could be dangerous or hazardous to the individual or others in the given location. Embodiments of risk aversion plans described herein are configurable for parameters, such as, but not limited to allergies, cardiovascular conditions, psychological conditions, avoidance of certain pathogens, etc.

Embodiments can provide the capability to generate new seating/room assignments for individuals based on the individuals' needs or providing an individual with a different route to a given location. In this regard the embodiments provide the individual the opportunity to avoid hazardous situations.

In some embodiments, a first user may be observed with a camera linked to a computer with 3-D facial recognition software. The first user may then be identified using the facial recognition software. Once the first user has been identified, the first user's health records can be retrieved by a computer connected to a server with a health record data base, provided the first subject has granted permission to access the health records. Further, a second user can be scanned using the same facial recognition identification system. The second user's health records can be accessed provided the second user has granted permission to access the records. The health records of the first and second user can be analyzed. If it is determined that a risk exists from the analysis of the health records an alert is sent to the first and second user.

For example, A first and second user have opted into a stand-alone application on their respective smartphones allowing for facial recognition scans to access their respective health records. A first user may have an O negative blood type (e.g., a universal blood donor). A second user with a rare blood type in the predetermined area may require a blood transfusion. An analysis of both user's health records will reveal that the first individual is able to donate blood to the second user, and an alert is sent to both users.

In an embodiment, the first user may receive an alert indicating that they could potentially help an individual by donating blood on the day the alert is received. Further, the second user may receive an alert after the first user does donate blood. In another embodiment, the first and second users may both receive an alert, simultaneously, which indicates that some action is requested, e.g., the first user could help an individual by donating blood and the second user should be in close proximity to a donation center, etc. It is noted that regardless of the embodiments presented above or discussed herein further, the first and second user may remain anonymous from one another.

In another example, multiple users enter a makeshift hospital during a local emergency. Each user, who volunteers to donate blood may have their face scanned which allows for a computer system to identify each user with facial recognition software. Once identified, each user's health records can be accessed and analyzed. A risk score can be calculated from the analysis (e.g., the risk score may be based on if the user meets the requirements for donating blood and/or what the blood type the user is). Finally, a volunteer list can be created from the health risk scores and users can be notified of their place in line to donate blood according to the volunteer list (e.g., users who meet the requirements to donate and are universal donors are drawn first, users who do not meet requirements are not allowed to be drawn, etc.).

In describing embodiments in detail with reference to the figures, it should be noted that references in the specification to “an embodiment,” “other embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, describing a particular feature, structure or characteristic in connection with an embodiment, one skilled in the art has the knowledge to affect such feature, structure or characteristic in connection with other embodiments whether or not explicitly described.

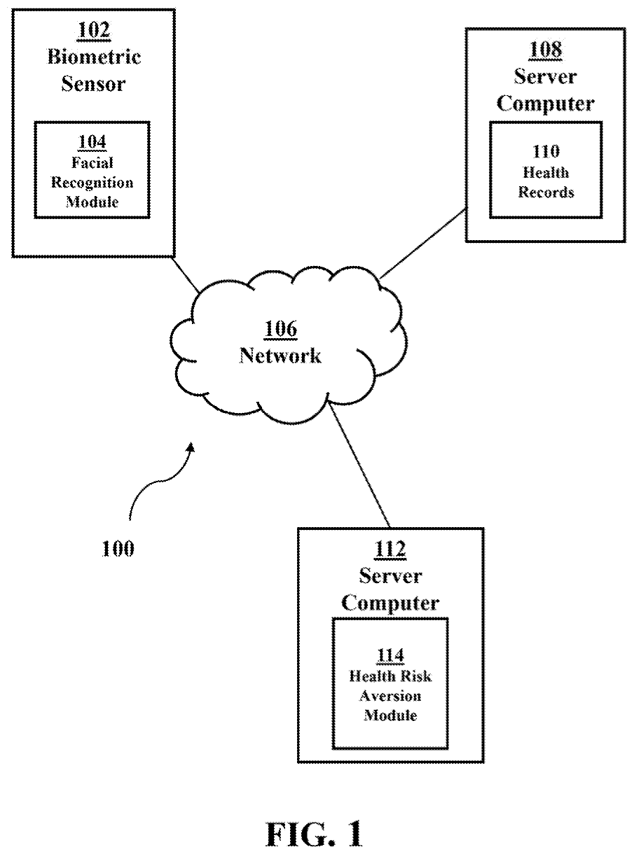

FIG. 1 is a functional block diagram illustrating, generally, an embodiment of a biometric identification risk aversion environment 100. The biometric identification risk aversion environment 100 comprises a facial recognition module 104 operational on a biometric sensor 102, a health records database 110 stored on a server computer 108, a health risk aversion module 114 operational on a server computer 112, and a network 106 supporting communications between biometric sensor 102, and server computers 108 and 112.

Biometric sensor 102 can be at least one camera, a retina scanner, fingerprint scanner or similar device suited to obtain the biometric information of an individual. The camera can operate in the visible spectrum, the infrared spectrum, or have the capability to capture thermal imagery. It should be noted, while facial recognition module 104 is shown to operate within biometric sensor 102 in FIG. 1, it may operate on a separate computing unit, such as server computer 112 or a general computing device, provided there is a communication channel between biometric senor 102 and facial recognition module 104, for example, but not limited to network 106 (discussed further below). Facial recognition module 104 may take an image, pixel data, or similar data extracted from biometric sensor 102 and analyze the facial features using a facial recognition technique, for example, but not limited to three-dimensional mapping, infrared mapping, or thermal infrared mapping. Further, in an embodiment, identification of individuals by facial recognition can be accomplished by a deep neural network (DNN) trained with three-dimensional mapping samples of human faces.

Network 106 can be, for example, a local area network (LAN), a wide area network (WAN) such as the Internet, or a combination of the two, and can include wired, wireless, or fiber optic connections. In general, network 106 can be any combination of connections and protocols that will support communications between biometric sensor 102 and server computers 108 and 112.

Server computers 108 and 112 can be a standalone computing device, management server, a web server, a mobile computing device, or any other electronic device or computing system capable of receiving, sending, and processing data. In other embodiments, server computers 108 and 112 can represent a server computing system utilizing multiple computers as a server system. In another embodiment, server computers 108 and 112 can be a laptop computer, a tablet computer, a netbook computer, a personal computer, a desktop computer, or any programmable electronic device capable of communicating with other computing devices (not shown) within biometric identification risk aversion environment 100 via network 106.

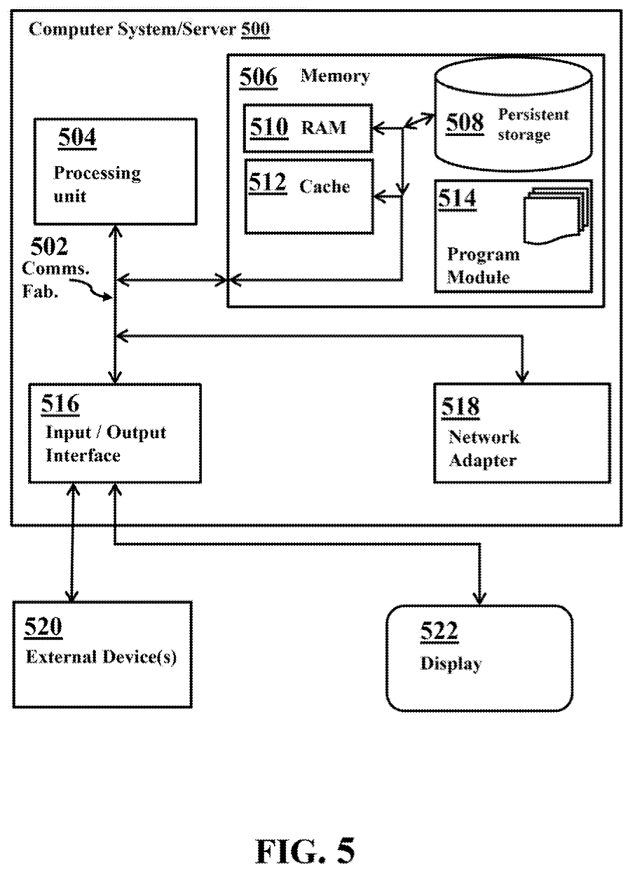

In another embodiment, server computers 108 and 112 represents a computing system utilizing clustered computers and components (e.g., database server computers, application server computers, etc.) that act as a single pool of seamless resources when accessed within biometric identification risk environment 100. Server computers 108 and 112 can include internal and external hardware components, as depicted and described in further detail in FIG. 5.

Health records 110 are shown to be stored on server computer 108, however health records 110 can be stored on any general computing device and on any persistent storage media (discussed further in FIG. 5) suitable for storing data. Health records can include, but are not limited to hospitalization records, medical records, immunization records, travel records, and pharmacy records. Further, the health records can be structured data or unstructured data. Further, if data is structured data, it may be included in a database format including, as a non-limiting example a Structured Query Language (SQL) database. Health records of individuals are only accessed upon a grant of permission by the individual in a way deemed legal by the local, state, or federal government.

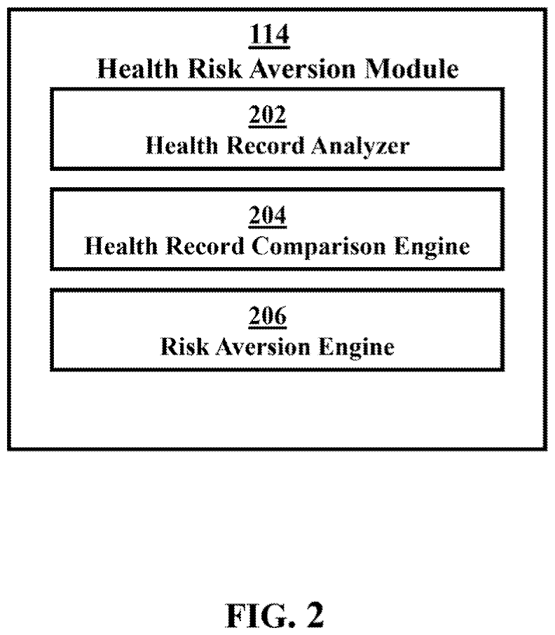

Health Risk Aversion Module (HRAM) 114 is operational on server 112. While HRAM 114 is shown on server 112, this is for illustrative purposes as it may be located on any general computing device with the ability to communicate with other devices, for a non-limiting example the device may be connected to network 106. HRAM 114 can provide the capability to extract data from health records, compare health records of individuals, create a risk aversion plan, and transmit a risk aversion plan to individuals, these capabilities are explained further below in FIG. 2.

Now with reference to FIG. 2, illustrated is a block diagram of the HRAM 114 comprising health record analyzer 202, health record comparison engine (HRCE) 204, and risk aversion engine 206.

Health record analyzer 202, of an embodiment of the present invention, can provide the capability to analyze health records (e.g., the health records 110 of FIG. 1). In some embodiments, health records 110 can be in structured form, for example SQL, can be extracted and input into HRCE 204. Health records in unstructured data form can analyzed using NLP models trained with preexisting health records. For example, but not limited to health professional voice patient notes or video recordings of a patent with health professional voice commentary. Additionally, in some embodiments, health record analyzer 202 can use optical character recognition (OCR) to analyze health records 110 in written form and send the output to the NLP. The NLP model can extract data from the output of the OCR and input the extracted data into HCRE 204.

HCRE 204, of an embodiment of the present invention, can provide the capability to parse through the health record data extracted from health record analyzer. For example, if an individual has recently been diagnosed with an illness and there is a record of said illness in the individual's health record. Meanwhile, there may be an individual that has not received a vaccination against that illness and has been identified in the same general location. If this scenario is discovered by the health record comparison engine, it can be transmitted to the risk aversion engine 206. In some embodiments, HCRE 204 can also be a DNN trained to determine if there is a correlation between an individual's condition and the health records using the output of the health record analyzer 202. For example, it is found in a user's health records that the user suffers from chronic fatigue, has food intolerances, and works night shifts. The DNN may find a correlation that implies that the user may have a vitamin D deficiency and suggest a vitamin D supplement or a sun lamp to the user. The DNN can then transmit the output to the health risk aversion engine 206.

In another embodiment of the invention, the data extracted by HRCE 204 can be converted into a health impact score for an individual when using the health data of two or more individuals. The health impact score can take into account many factors for example, medical conditions, allergies, blood pressure, age, body mass index, medications, and psychological conditions. For example, when using two values to calculate a health impact score, a correlation coefficient (r value) may be the health impact score, each of these conditions can be used, using a baseline, such as, the ideal blood pressure, body mass index, or the severity of an allergy. The equation for and correlation coefficient may be as follows:

$$r = \frac{1}{n - 1} \sum ( \frac{{x}_{i} - \overline{x}}{{s}_{x}} ) ( \frac{{y}_{i} - \overline{y}}{{s}_{y}} )$$

Where r is the correlation coefficient, xᵢ is the is the sample for the first condition, x̅ is the mean of the condition for the sample population, sₓ is the known standard deviation for that condition, yᵢ is the is the sample for the second condition, y̅ is the mean of the second condition for the sample population, s_{y} is the known standard deviation for that condition, and n is the sample size.

There may be other ways to calculate a health impact score and it should be understood that the correlation coefficient is only a non-limiting example. In an embodiment HCRE 204 will determine whether from parsing through the health record data or calculating a health risk score if there is a potential health risk and transmit the potential health risk to the Risk aversion engine. Health risks can also include but are not limited to emergencies in the general vicinity of the person.

Risk aversion engine 206 of an embodiment may receive the input from HCRE 204 if there is a potential health risk. Risk aversion engine can provide the capability to change seating arrangements or divert individuals to different areas when a health risk is detected. Embodiments of risk aversion engine can take many different models, for example, but not limited to a DNN that is trained with seating information may prevent peanuts from being served in an area where there is an individual with a known severe peanut allergy. In another embodiment, seating assignments at a concert venue may be reassigned to allow for persons with hearing sensitivity to be seated closer/farther from speakers. In another embodiment, a DNN with light sensitivity may be alerted that the movie they about to watch has multiple instances of flashing lights. Risk aversion engine can also provide the capability to transmit new travel itinerary to a person's mobile electronic device, through e-mail, standard messaging systems, or mobile application suitable for transfer of such data.

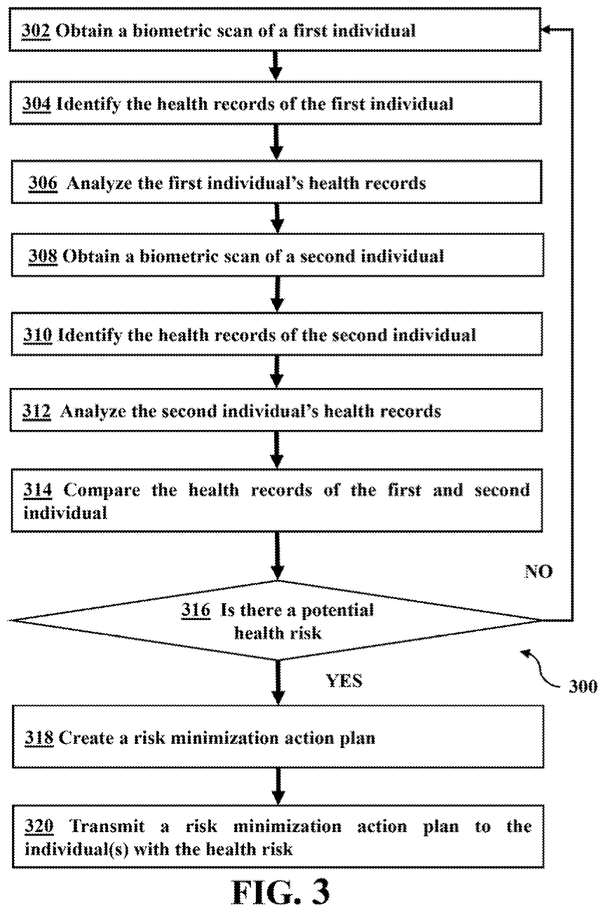

FIG. 3 is a flowchart depicting the operational steps of risk aversion by biometric identification method 300 according to an embodiment of the invention. At step 302, obtain a biometric scan of a first individual (e.g., user) utilizing biometric sensor 102. Next, at step 304 identify the health records of the first individual using facial recognition module 104. Next, at step 306 analyze the health records of the first individual with health record analyzer 202. Next, at step 308, obtain a biometric scan of a second individual utilizing biometric sensor 102. Next, at step 310 identify the health records of the second individual using facial recognition module 104. Next, at step 312 analyze the health records of the second individual with health record analyzer 202. Next, at step 314 compare the health records of the first and second individual with HRCE 204. Next at step 316, determine if there is a potential health risk using HRCE 204. In some embodiments, the determining of a potential health risk may include or be based on if a risk threshold was met. If there is a health risk (e.g., the risk threshold is met or exceeded), at step 318, create a risk minimization action plan (e.g., an alert) with health risk aversion engine 206. Next, at step 320 transmit (e.g., display) risk minimization action plan to the individual with the health risk with risk aversion engine 206.

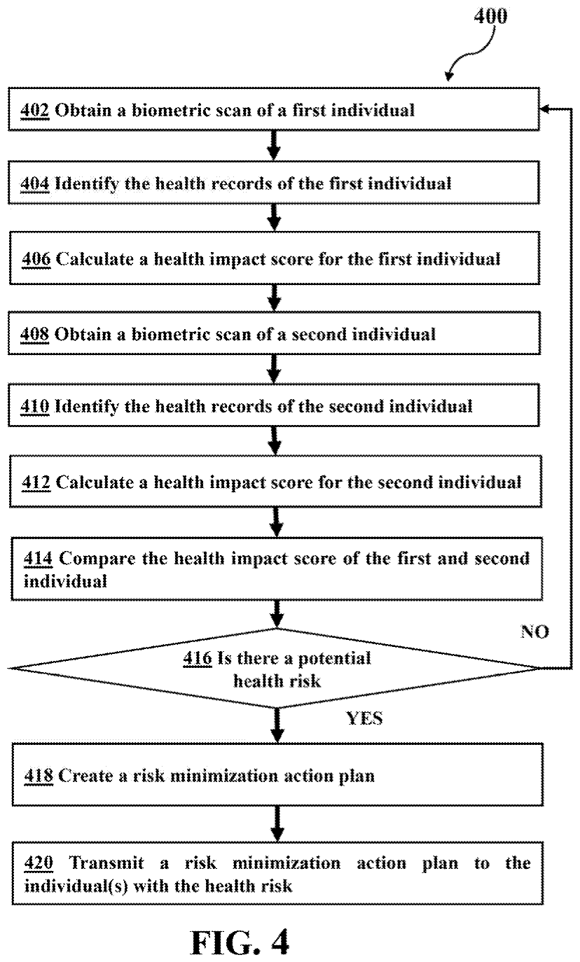

FIG. 4 is a flowchart depicting the operational steps of risk aversion by biometric identification method 400, according to an embodiment of the invention. At step 402, obtain a biometric scan of a first individual utilizing biometric sensor 102. Next, at step 404 identify the health records of the first individual using facial recognition module 104. Next, at step 406 calculate a health impact (e.g., risk) score for the first individual with health record analyzer 202. Next, at step 408, obtain a biometric scan of a second individual utilizing biometric sensor 102. Next, at step 410 identify the health records of the second individual using facial recognition module 104. Next, at step 412 calculate a health impact (e.g., risk) score for the second individual with health record analyzer 202. Next, at step 413 compare the health impact scores of the first and second individual with HCRE 204. Next at step 416, determine if there is a potential health risk using HCRE 204. If there is a health risk, at step 418, create a risk minimization action plan with health risk aversion engine 206. Next, at step 420 transmit risk minimization action plan to the individual with the higher health risk with risk aversion engine 206.

FIG. 5 depicts computer system 500, which is representative of a device within health risk biometric identification risk aversion environment 100. Computer system 500 includes communications fabric 502, which provides communications between computer processor(s) 504, memory 506, persistent storage 508, network adaptor 518, and input/output (I/O) interface(s) 516. Communications fabric 502 can be implemented with any architecture designed for passing data and/or control information between processors (such as microprocessors, communications and network processors, etc.), system memory, peripheral devices, and any other hardware components within a system. For example, communications fabric 502 can be implemented with one or more buses.

Computer system 500 includes processors 504, cache 512, memory 506, persistent storage 508, network adaptor 518, input/output (I/O) interface(s) 516 and bus 502. Communications fabric 502 provides communications between cache 512, memory 506, persistent storage 508, network adaptor 518, and input/output (I/O) interface(s) 516. Communications fabric 502 can be implemented with any architecture designed for passing data and/or control information between processors (such as microprocessors, communications and network processors, etc.), system memory, peripheral devices, and any other hardware components within a system. For example, communications fabric 502 can be implemented with one or more buses or a crossbar switch.

Memory 506 and persistent storage 508 are computer readable storage media. In this embodiment, memory 506 includes random access memory (RAM) 510. In general, memory 506 can include any suitable volatile or non-volatile computer readable storage media. Cache 512 is a fast memory that enhances the performance of processors 504 by holding recently accessed data, and data near recently accessed data, from memory 506.

Program instructions and data used to practice embodiments of the present invention may be stored in persistent storage 508 and in memory 506 for execution by one or more of the respective processors 504 via cache 512. In an embodiment, persistent storage 508 includes a magnetic hard disk drive. Alternatively, or in addition to a magnetic hard disk drive, persistent storage 508 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 508 may also be removable. For example, a removable hard drive may be used for persistent storage 508. Other examples include optical and magnetic disks, thumb drives, and smart cards that are inserted into a drive for transfer onto another computer readable storage medium that is also part of persistent storage 508.

Network adaptor 518, in these examples, provides for communications with other data processing systems or devices. In these examples, network adaptor 518 includes one or more network interface cards. Network adaptor 518 may provide communications through the use of either or both physical and wireless communications links. Program instructions and data used to practice embodiments of the present invention may be downloaded to persistent storage 508 through network adaptor 518.

I/O interface(s) 516 allows for input and output of data with other devices that may be connected to each computer system. For example, I/O interface 516 may provide a connection to external devices 520 such as a keyboard, keypad, a touch screen, and/or some other suitable input device. External devices 520 can also include portable computer readable storage media such as, for example, thumb drives, portable optical or magnetic disks, and memory cards. Software and data used to practice embodiments of the present invention can be stored on such portable computer readable storage media and can be loaded onto persistent storage 508 via I/O interface(s) 516. I/O interface(s) 516 also connect to display 522.

Display 520 provides a mechanism to display data to a user and may be, for example, a computer monitor or projector.

The components described herein are identified based upon the application for which they are implemented in a specific embodiment of the invention. However, it should be appreciated that any particular component nomenclature herein is used merely for convenience, and thus the invention should not be limited to use solely in any specific application identified and/or implied by such nomenclature.

The present invention may be a system, a method and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems) and computer program products according to embodiments of the invention. It is understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality and operation of possible implementations of systems, methods, and computer program products according to various embodiments of the present invention. In this regard, each block in the flowchart or block diagrams may represent a module, segment or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

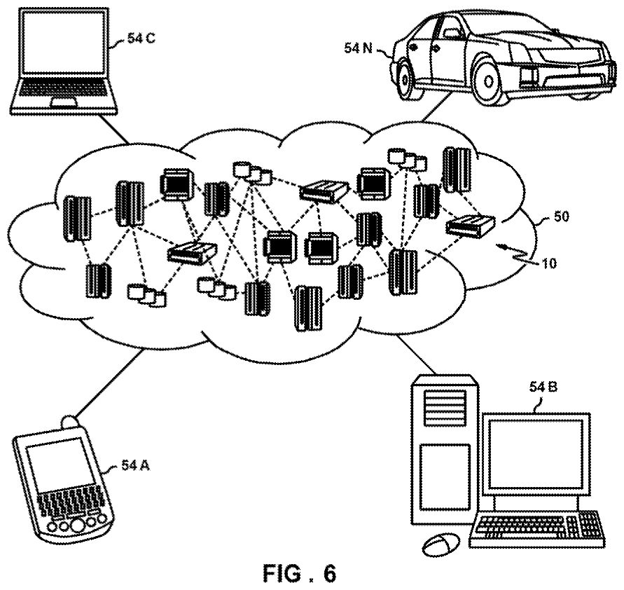

FIG. 6 is a block diagram depicting a cloud computing environment 50 in accordance with at least one embodiment of the present invention. Cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

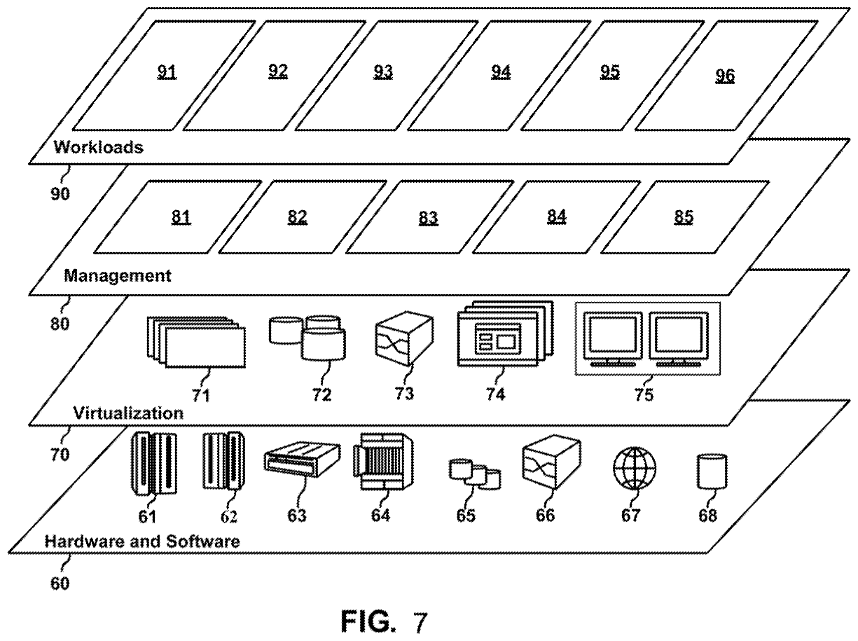

FIG. 7 is a block diagram depicting a set of functional abstraction model layers provided by cloud computing environment 50 depicted in FIG. 6 in accordance with at least one embodiment of the present invention. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and risk aversion by biometric identification 96.

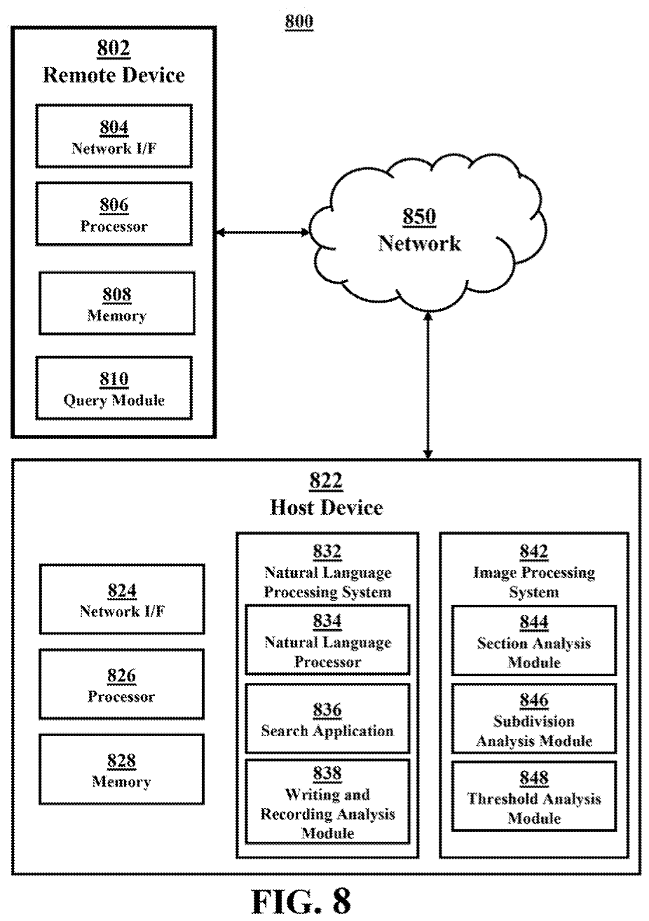

Turning now to FIG. 8, illustrated is a block diagram of an example computing environment 800 in which illustrative embodiments of the present disclosure may be implemented. In some embodiments, the computing environment 800 may include a remote device 802 and a host device 822.

Consistent with various embodiments, the host device 822 and the remote device 802 may be computer systems. The remote devices 802 and the host device 822 may include one or more processors 806 and 826 and one or more memories 808 and 828, respectively. The remote device 802 and the host device 822 may be configured to communicate with each other through an internal or external network interface 804 and 824. The network interfaces 804 and 824 may be modems or network interface cards. The remote device 802 and/or the host device 822 may be equipped with a display or monitor. Additionally, the remote device 802 and/or the host device 822 may include optional input devices (e.g., a keyboard, mouse, scanner, or other input device), and/or any commercially available or custom software (e.g., browser software, communications software, server software, natural language processing software, search engine and/or web crawling software, filter modules for filtering content based upon predefined parameters, etc.). In some embodiments, the remote device 802 and/or the host device 822 may be servers, desktops, laptops, or hand-held devices.

The remote device 802 the host device 822 may be distant from each other and communicate over a network 850. In some embodiments, the host device 822 may be a central hub from which remote device 802 can establish a communication connection, such as in a client-server networking model. Alternatively, the host device 822 and remote device 802 may be configured in any other suitable networking relationship (e.g., in a peer-to-peer configuration or using any other network topology).

In some embodiments, the network 850 can be implemented using any number of any suitable communications media. For example, the network 850 may be a wide area network (WAN), a local area network (LAN), an internet, or an intranet. In certain embodiments, the remote device 802 and the host device 822 may be local to each other, and communicate via any appropriate local communication medium. For example, the remote device 802 and the host device 822 may communicate using a local area network (LAN), one or more hardwire connections, a wireless link or router, or an intranet. In some embodiments, the remote device 802 and the host device 822 may be communicatively coupled using a combination of one or more networks and/or one or more local connections. For example, the remote device 802 may be hardwired to the host device 822 (e.g., connected with an Ethernet cable) or the remote device 802 may communicate with the host device using the network 850 (e.g., over the Internet). In some embodiments the network 850 may be the same network 106 of FIG. 1 or in communication with network 106.

In some embodiments, the network 850 can be implemented within a cloud computing environment, or using one or more cloud computing services, such as those discussed in regard to FIGS. 6-7. Consistent with various embodiments, a cloud computing environment may include a network-based, distributed data processing system that provides one or more cloud computing services. Further, a cloud computing environment may include many computers (e.g., hundreds or thousands of computers or more) disposed within one or more data centers and configured to share resources over the network 850.

In some embodiments, the remote device 802 may enable a user to input (or may input automatically with or without a user) a query/request to the host device 822 in order to identify portions of records containing unstructured data. For example, the remote device 802 may include a query module 810 and a user interface (UI). The query module 810 may be in the form of a web browser or any other suitable software module, and the UI may be any type of interface (e.g., command line prompts, menu screens, graphical user interfaces). The UI may allow a user to interact with the remote device 802 to input, using the query module 810, a query to the host device 822, which may receive the query.

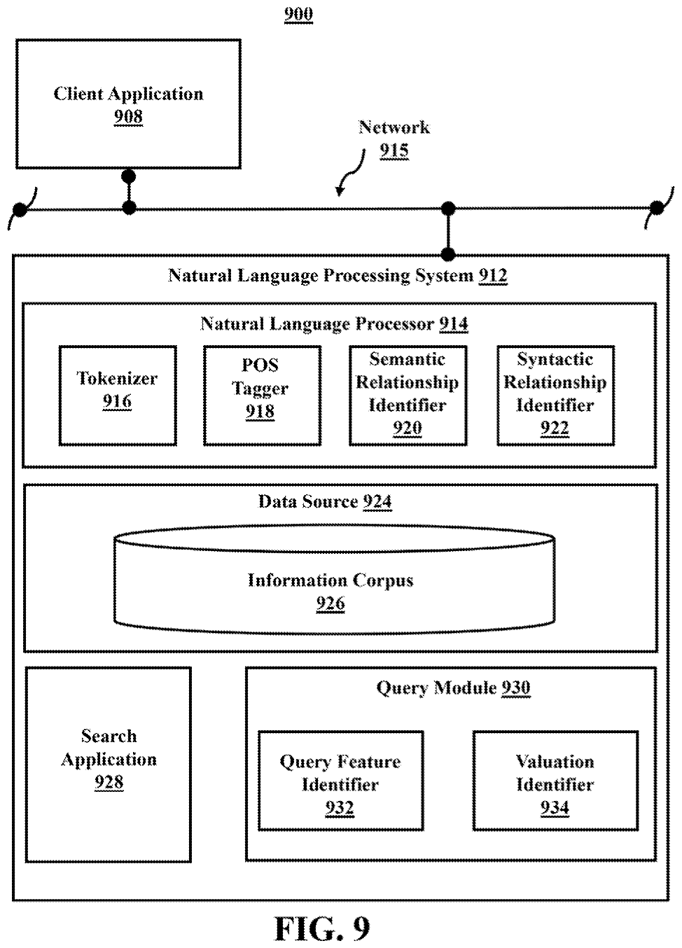

In some embodiments, the host device 822 may include a natural language processing system 832. The natural language processing system 832 may include a natural language processor 834, a search application 836, and a recording and writing analysis module 838. The natural language processor 834 may include numerous subcomponents, such as a tokenizer, a part-of-speech (POS) tagger, a semantic relationship identifier, and a syntactic relationship identifier. An example natural language processor is discussed in more detail in reference to FIG. 9.

The search application 836 may be implemented using a conventional or other search engine and may be distributed across multiple computer systems. The search application 836 may be configured to search one or more databases (e.g., repositories) or other computer systems for content that is related to a query submitted by the remote device 802. For example, the search application 836 may be configured to search medical dictionaries, papers, and/or archived medical reports to help identify a particular subject related to a query provided for a health record. The writing and recording analysis module 838 may be configured to analyze a health record to identify a particular subject (e.g., symptoms of diabetes, etc.). The writing and recording analysis module 838 may include one or more modules or units, and may utilize the search application 836, to perform its functions, as discussed in more detail in reference to FIG. 9.

In some embodiments, the host device 822 may include an image processing system 842. The image processing system 842 may be configured to analyze unstructured data of a health record to create an image analysis (for example, scanned laboratory results, scanned printed medical procedure results, or hand-written notes of a healthcare professional). The image processing system 842 may utilize one or more models, modules, or units to perform its functions (e.g., to analyze the images associated with the health record and generate an image analysis). For example, the image processing system 842 may include one or more image processing models that are configured to identify handwritten notes of healthcare professionals in a user health record. The image processing models may include a section analysis module 844 to analyze single images associated with the record and to identify one or more features of the single images. As another example, the image processing system 842 may include a subdivision analysis module 846 to group multiple health records together identified to have a common feature of the one or more features (e.g., separating health records associated with head pain into two groups: one group that indicates headaches and one indicating migraines, etc.). In some embodiments, the image processing models may be implemented as software modules. For example, the image processing system 842 may include a section analysis module and a subdivision analysis module. In some embodiments, a single software module may be configured to analyze the health records using the image processing models.

In some embodiments, the image processing system 842 may include a threshold analysis module 848. The threshold analysis module 848 may be configured to compare, the instances of a particular health condition identified in a subdivision of sections of the health record against a threshold number of instances. The threshold analysis module 848 may then determine if the subdivision should be considered by the health risk aversion module 114.

In some embodiments, the host device may have an optical character recognition (OCR) module. The OCR module may be configured to receive a health record sent from the remote device 802 and perform optical character recognition (or a related process) on the health record to convert it into machine-encoded text so that the natural language processing system 832 may perform NLP on the report. For example, the remote device 802 may transmit a video of a medical procedure to the host device 822. The OCR module may convert the unstructured data in health records into machine-encoded text, and then the converted unstructured data in health records may be sent to the natural language processing system 832 for analysis. In some embodiments, the OCR module may be a subcomponent of the natural language processing system 832. In other embodiments, the OCR module may be a standalone module within the host device 822. In still other embodiments, the OCR module may be located on the remote device 802 and may perform OCR on the health record before the health record is sent to the host device 822.

While FIG. 8 illustrates a computing environment 800 with a single host device 822 and a remote device 802, suitable computing environments for implementing embodiments of this disclosure may include any number of remote devices and host devices. The various models, modules, systems, and components illustrated in FIG. 8 may exist, if at all, across a plurality of host devices and remote devices. For example, some embodiments may include two host devices. The two host devices may be communicatively coupled using any suitable communications connection (e.g., using a WAN, a LAN, a wired connection, an intranet, or the Internet). The first host device may include a natural language processing system configured to receive and analyze a voice data associated with a health record, and the second host device may include an image processing system configured to receive and analyze unstructured data associated with a health record, to generate an image analysis.

It is noted that FIG. 8 is intended to depict the representative major components of an exemplary computing environment 800. In some embodiments, however, individual components may have greater or lesser complexity than as represented in FIG. 8, components other than or in addition to those shown in FIG. 8 may be present, and the number, type, and configuration of such components may vary.

Referring now to FIG. 9, shown is a block diagram of an exemplary system architecture 900, including a natural language processing system 912, configured to analyze medical data to identify a condition and a criterion, in accordance with embodiments of the present disclosure. In some embodiments, a remote device (such as remote device 802 of FIG. 8) may submit a health record to be analyzed to the natural language processing system 912 which may be housed on a host device (such as host device 822 of FIG. 8). Such a remote device may include a client application 908, which may itself involve one or more entities operable to generate or modify information associated with the health record that is then dispatched to a natural language processing system 912 via a network 915.

Consistent with various embodiments, the natural language processing system 912 may respond to health record submission sent by a client application 908. Specifically, the natural language processing system 912 may analyze a received a (e.g., voice, written, image, etc.) health record related to the health record to identify a particular subject in the health record. In some embodiments, the natural language processing system 912 may include a natural language processor 914, data sources 924, a search application 928, and a query module 930. The natural language processor 914 may be a computer module that analyzes the health record. The natural language processor 914 may perform various methods and techniques for analyzing health record (e.g., syntactic analysis, semantic analysis, etc.). The natural language processor 914 may be configured to recognize and analyze any number of natural languages. In some embodiments, the natural language processor 914 may group one or more sections of a recording into one or more subdivisions. Further, the natural language processor 914 may include various modules to perform analyses of health record. These modules may include, but are not limited to, a tokenizer 916, a part-of-speech (POS) tagger 918 (e.g., which may tag each of the one or more sections in which the particular subject is identified), a semantic relationship identifier 920, and a syntactic relationship identifier 922.

In some embodiments, the tokenizer 916 may be a computer module that performs lexical analysis. The tokenizer 916 may convert a sequence of characters (e.g., images, sounds, etc.) into a sequence of tokens. A token may be a string of characters included in a recording and categorized as a meaningful symbol. Further, in some embodiments, the tokenizer 916 may identify word boundaries in a health record and break any text within the health record (e.g., from hand-written notes, scanned documents, etc.) into their component text elements, such as words, multiword tokens, numbers, and punctuation marks. In some embodiments, the tokenizer 916 may receive a string of characters, identify the lexemes in the string, and categorize them into tokens.

Consistent with various embodiments, the POS tagger 918 may be a computer module that marks up a word in a health record to correspond to a particular part of speech. The POS tagger 918 may read a passage or other text in natural language and assign a part of speech to each word or other token. The POS tagger 918 may determine the part of speech to which a word (or other spoken element) corresponds based on the definition of the word and the context of the word. The context of a word may be based on its relationship with adjacent and related words in a phrase, sentence, or paragraph. In some embodiments, the context of a word may be dependent on one or more previously analyzed health records (e.g., the content of one record may shed light on the meaning of one or more subjects in another record). Examples of parts of speech that may be assigned to words include, but are not limited to, nouns, verbs, adjectives, adverbs, and the like. Examples of other part of speech categories that POS tagger 918 may assign include, but are not limited to, comparative or superlative adverbs, wh-adverbs, conjunctions, determiners, negative particles, possessive markers, prepositions, wh-pronouns, and the like. In some embodiments, the POS tagger 918 may tag or otherwise annotate tokens of a recording with part of speech categories. In some embodiments, the POS tagger 918 may tag tokens or words of a recording to be parsed by the natural language processing system 912.

In some embodiments, the semantic relationship identifier 920 may be a computer module that may be configured to identify semantic relationships of recognized subjects (e.g., words, phrases, images, etc.) in a recording. In some embodiments, the semantic relationship identifier 920 may determine functional dependencies between entities and other semantic relationships.

Consistent with various embodiments, the syntactic relationship identifier 922 may be a computer module that may be configured to identify syntactic relationships in a health record composed of tokens. The syntactic relationship identifier 922 may determine the grammatical structure of sentences such as, for example, which groups of words are associated as phrases and which word is the subject or object of a verb. The syntactic relationship identifier 922 may conform to formal grammar.

In some embodiments, the natural language processor 914 may be a computer module that may group sections of a health record into subdivisions and generate corresponding data structures for one or more subdivisions of the health record. For example, in response to receiving a recording at the natural language processing system 912, the natural language processor 914 may output subdivisions of the recording as data structures. In some embodiments, a subdivision may be represented in the form of a graph structure. To generate the subdivision, the natural language processor 914 may trigger computer modules 916-922.

In some embodiments, the output of natural language processor 914 may be used by search application 928 to perform a search of a set of (i.e., one or more) corpora to retrieve one or more subdivision including a particular subject associated with a query and send the output to an image processing system. As used herein, a corpus may refer to one or more data sources, such as the data sources 924 of FIG. 9. In some embodiments, the data sources 924 may include video libraries, data warehouses, information corpora, data models, and document repositories. In some embodiments, the data sources 924 may include an information corpus 926. The information corpus 926 may enable data storage and retrieval. In some embodiments, the information corpus 926 may be a subject repository that houses a standardized, consistent, clean, and integrated list of images and dialogue. For example, the information corpus 926 may include medical journals and medical records. The data may be sourced from various operational systems. Data stored in the information corpus 926 may be structured in a way to specifically address reporting and analytic requirements. In some embodiments, the information corpus 926 may be a relational database.

In some embodiments, the query module 930 may be a computer module that identifies common features within sections of a recording and a particular subject of a query in subdivisions of sections of the recording. In some embodiments, the query module 930 may include a query feature identifier 932 and a valuation identifier 934. When a query is received by the natural language processing system 912, the query module 930 may be configured to analyze a health record using natural language processing to identify a particular subject. The query module 930 may first identity one or more subjects in the recording using the natural language processor 914 and related subcomponents 916-922. After identifying the one or more subjects, the query feature identifier 932 may identify one or more common features present in sections of the health record. In some embodiments, the common features in the sections may be the same subject that is identified (e.g., all of the analyzed health records include information associated with vitamin D deficiencies, etc.). Once a common feature is identified, the query feature identifier 932 may be configured to transmit the sections that include the common feature to an image processing system (shown in FIG. 8). After identifying common features of a health record using the query feature identifier 932, the query module may group sections of the health record having common features into subdivisions. The valuation identifier 934 may then identify a particular subject in subdivisions of the health record. After identifying a particular subject relating to the query, the valuation identifier 934 may be configured to transmit the criterion to an image processing system (shown in FIG. 8). In some embodiments, the valuation identifier 934 evaluations and/or determines if a particular subject meets a valuation threshold. If the evaluation identifier 934 determines that a particular subject meets the valuation threshold, the particular subject may be sent to the processing system, which may then forward the particular subject to be used by any of the various embodiments described throughout the present disclosure.

Although the present disclosure has been described in terms of specific embodiments, it is anticipated that alterations and modification thereof will become apparent to the skilled in the art. Therefore, it is intended that the following claims be interpreted as covering all such alterations and modifications as fall within the true spirit and scope of the disclosure.

What is claimed is:

1. A computer-implemented method for identifying health risks associated with a user, the method comprising:
obtaining, by one or more processors, a biometric scan of a first user and a second user, wherein the first user and the second user are within a predetermined geographical location;
identifying one or more records associated with the first user and one or more records associated with the second user based on the biometric scan;
analyzing the one or more records associated with the first user and the one or more records associated with the second user;
determining, from the analyzing, whether a risk threshold is exceeded; and
displaying an alert to the first user and the second user.

2. The method of claim 1, wherein determining whether a risk threshold is exceeded includes:
comparing the one or more records associated with the first user to the one or more records of the second user; and
identifying that the first user has a susceptibility that is uncomplimentary to the second user, wherein identifying that the susceptibility is uncomplimentary indicates that the risk threshold is exceeded.

3. The method of claim 2, further comprising:
responsive, to identifying the risk threshold is exceeded, creating, by the one or more processors, a risk minimization action plan; and
transmitting the risk minimization action plan to the first user as the alert.

4. The method of claim 1, further comprising:
comparing the one or more records associated with the first user to the one or more records of the second user;
identifying that the first user has a susceptibility that is complimentary to the second user, wherein identifying that the susceptibility is complimentary indicates that the risk threshold is not exceeded; and
transmitting, as the alert, a message to both the first user and the second user, wherein the message indicates that there is no potential risk in the geographical location.

5. The method of claim 4, further comprising:
notifying, by the one or more processors, the second user via a mobile electronic device of the second user of an opportunity to assist the first user.

6. The method of claim 1, wherein determining whether a risk threshold is exceeded includes:
calculating, by the one or more processors, a risk score from the analysis of the one or more records respectively associated with the first and second users.

7. The method of claim 6, further comprising:
identifying that the first user has a higher risk score than the second individual; and
transmitting to a mobile electronic device of the first user a risk minimization action plan as the alert.

8. A computer program product comprising a computer readable storage medium having program instructions embodied therewith, the program instructions executable by a processor to cause the processors to perform a function, the function comprising:
obtaining a biometric scan of a first user and a second user, wherein the first user and the second user are within a predetermined geographical location;
identifying one or more records associated with the first user and one or more records associated with the second user based on the biometric scan;
analyzing the one or more records associated with the first user and the one or more records associated with the second user;
determining, from the analyzing, whether a risk threshold is exceeded; and
displaying an alert to the first user and the second user.

9. The computer program product of claim 8, wherein determining whether a risk threshold is exceeded includes:
comparing the one or more records associated with the first user to the one or more records of the second user; and
identifying that the first user has a susceptibility that is uncomplimentary to the second user, wherein identifying that the susceptibility is uncomplimentary indicates that the risk threshold is exceeded.

10. The computer program product of claim 9, further comprising:
responsive, to identifying the risk threshold is exceeded, creating, by the one or more processors, a risk minimization action plan; and
transmitting the risk minimization action plan to the first user as the alert.

11. The computer program product of claim 8, further comprising:
comparing the one or more records associated with the first user to the one or more records of the second user;
identifying that the first user has a susceptibility that is complimentary to the second user, wherein identifying that the susceptibility is complimentary indicates that the risk threshold is not exceeded; and
transmitting, as the alert, a message to both the first user and the second user, wherein the message indicates that there is no potential risk in the geographical location.

12. The computer program product of claim 11, further comprising:
notifying, by the one or more processors, the second user via a mobile electronic device of the second user of an opportunity to assist the first user.

13. The computer program product of claim 8, wherein determining whether a risk threshold is exceeded includes:
calculating, by the one or more processors, a risk score from the analysis of the one or more records respectively associated with the first and second users.

14. The computer program product of claim 13, further comprising:
identifying that the first user has a higher risk score than the second individual; and
transmitting to a mobile electronic device of the first user a risk minimization action plan as the alert.

15. A system comprising:
a memory; and
a processor in communication with the memory, the processor being configured to perform operations comprising:
obtaining, by one or more processors, a biometric scan of a first user and a second user, wherein the first user and the second user are within a predetermined geographical location;
identifying one or more records associated with the first user and one or more records associated with the second user based on the biometric scan;
analyzing the one or more records associated with the first user and the one or more records associated with the second user;
determining, from the analyzing, whether a risk threshold is exceeded; and
displaying an alert to the first user and the second user.

16. The system of claim 15, wherein the operations further comprise:
comparing the one or more records associated with the first user to the one or more records of the second user; and
identifying that the first user has a susceptibility that is uncomplimentary to the second user, wherein identifying that the susceptibility is uncomplimentary indicates that the risk threshold is exceeded.

17. The system of claim 16, wherein the operations further comprise:
responsive, to identifying the risk threshold is exceeded, creating, by the one or more processors, a risk minimization action plan.

18. The system of claim 15, wherein the operations further comprise:
comparing the one or more records associated with the first user to the one or more records of the second user.

19. The system of claim 18, wherein the operations further comprise:
notifying, by the one or more processors, the second user via a mobile electronic device of the second user of an opportunity to assist the first user.

20. The system of claim 15, wherein determining whether a risk threshold is exceeded includes:
calculating, by the one or more processors, a risk score from the analysis of the one or more records respectively associated with the first and second users.
